(19) United States
(12) Patent Application Publication
(10) Pub. No.: US 2022/0076826 A1
(43) Pub. Date: Mar. 10, 2022

(54) MODEL BASED ON MACHINE LEARNING-RADIOMICS AND APPLICATION THEREOF

(71) Applicants: Fanxin Zeng, Dazhou (CN); Fanwei Zeng, Dazhou (CN); Jing Zhu, Dazhou (CN); Qianrong Xie, Dazhou (CN); Pingxi Wang, Dazhou (CN); Yue Chen, Dazhou (CN)

(72) Inventors: Fanxin Zeng, Dazhou (CN); Fanwei Zeng, Dazhou (CN); Jing Zhu, Dazhou (CN); Qianrong Xie, Dazhou (CN); Pingxi Wang, Dazhou (CN); Yue Chen, Dazhou (CN)

(21) Appl. No.: 17/013,661

(22) Filed: Sep. 7, 2020

(51) Int. Cl. G16H 50/20; G16H 50/30

(52) U.S. Cl. CPC G16H 50/20; G16H 50/30

(57) ABSTRACT

The invention discloses a model based on machine learning-radiomics which is a Nomogram model; the establishment of said Nomogram model comprising following steps: S1. collecting data and predicting factor selection; S2. combining selected 6 radiomics characteristics according to contribution weighting, developing a rad-score model; S3. establishing different diagnosis models, comparing performances of the diagnosis models in the diagnosis of osteoporosis and osteopenia, wherein said diagnosis models comprise: Clinics model, Radiomics model, and Combined model; S4. performing visualization processing on combined model by using the “rms” packet in R software to obtain Nomogram model; S5. validation of the Nomogram model. The invention combines characteristics of the radiomics and clinical risk factors to establish a combined Nomogram model to diagnose conditions of osteoporosis and osteopenia of patients. The diagnosis method can accurately distinct patients with osteoporosis from patients with osteopenia, having great application value for selection of clinical therapeutic regimen.

### BACKGROUND OF THE INVENTION

### 1. Field of the Invention

The invention relates to the field of medical technology, specifically relating to a model based on machine learning-radiomics and application thereof.

### 2. Description of the Related Art

Osteoporosis, a main musculoskeletal disease, features in bone mass loss which destroys the microstructure of the skeleton, increases the fragility of the skeleton, and raises the susceptibility of fracture. Fracture caused by osteoporosis poses tremendous burden on the world, while finding the bone mass loss at its early stage and timely intervention can alleviate the burden of the osteoporosis on the economy and patients' body and mind. At present, dual-energy x-ray (DXA) is widely used in the diagnosis of osteoporosis, but it merely reflects the mean density of ateral radiograph which may be interfered by angiosteosis, osteophyte and posture. Quantitative computed tomography (QCT) is an imaging technique based on absorption of radiation to measure the volumetric bone mineral density, respectively conducting evaluation on the cortical bone and cancellous bone. Changes in bone mineral density measured by QCT are more sensitive to age-related or treatment-related properties of the entire vertebral body than measured by DXA.

Since the proposal of Radiomics in 2012, a high-throughput method for extracting specific image features from standard medical images has attracted an increased attention. Radiomics has been successfully applied to the prediction and differentiation of non-small cell lung cancer distant metastasis, colorectal carcinoma lymph node metastasis, gastric carcinoma tumor types, multiple myeloma types. The characteristics of the radiomics were introduced into a skeletal muscle system for the first time by Tagliafico AS and the like, and the bone tissue of multiple myeloma is divided into a focal mode and a diffuse mode, wherein the validity of model validation reaches 73% and 71% respectively. However, currently, the differential model of osteoporosis and osteopenia based on QCT images has not been established.

### SUMMARY OF THE INVENTION

To solve above problems in prior art, the invention provides a model based on machine learning-radiomics and application thereof. The invention adopts the following technical solution to realized the purpose.

A model based on machine learning-radiomics, which is the Nomogram model;

the establishment of said Nomogram model comprises following steps:

S1. collecting data and predicting factor selection: selecting case samples, and processing data by using machine learning minimum redundancy and maximum relevance (mRMR) algorithm and least absolute shrinkage and selection operator (LASSO) algorithm via “mRMRe” and “glmnet” packet in R software;

20 potential predictors are selected from 851 radiomics characteristics via the minimum redundancy and maximum relevance (mRMR) algorithm;

the optimal characteristics of 6 non-zero coefficients are further selected by using least absolute shrinkage and selection operator (LASSO) algorithm;

selecting candidate factors of clinical information by logistic regression, and screening out 3 clinical characteristics: Age, alkaline phosphatase (ALP), and homocysteine (HCY);

S2. combining the selected 6 radiomics characteristics according to contribution weighting, and developing rad-score model;

S3. establishing different diagnosis models, and comparing the performances of the diagnosis models in the diagnosis of osteoporosis and osteopenia, wherein said diagnosis models comprise:

Clinics model, built merely by 3 clinical characteristics (Age, ALP, HCY), and the AUC in the training cohorts is 0.81 (95% CI, 0.78-0.86) while the AUC in the validation cohorts is 0.79 (95% CI, 0.71-0.86);

Radiomics model, built merely by rad-score and the AUC in the training cohorts is 0.96 (95% CI, 0.94-0.98) while the AUC in the validation cohorts is 0.96 (95% CI, 0.92-1.00);

Combined model, built by combination of rad-score and 3 clinical characteristics (Age, ALP, HCY) wherein the AUC in the training cohorts is 0.96 (95% CI, 0.95-0.98) while the AUC in the validation cohorts is 0.96 (95% CL 0.92-1.00);

S4. performing visualization processing on the combined model by using the “rms” packet in the R software to obtain the Nomogram model;

S5. Validation of the Nomogram model.

Preferably, the case samples in step S1 are more than or equal to 300 cases; the cases' selection adopts an exclusive method, and the exclusion criteria are as follows: a. fracture of lumbar vertebra or internal fixation of fracture of lumbar vertebra; b. malignant space-occupying lesion of lumbar vertebra; c. metabolic or endocrine diseases such as hyperthyroidism or hypothyroidism, space-occupying lesions of thyroid, diabetes, neurological diseases (e.g., parkinson's disease, alzheimer's disease); d. chronic obstructive pulmonary disease; e. poor image quality; f. the clinical characteristics of the missed diagnosis are more than 3.

An application of said model based on machine learning-radiomics for diagnosing and identifying osteoporosis and osteopenia.

The invention has following advantageous effects:

The invention has established and verified a bone mass individualized diagnosis model based on QCT images, wherein the model is a large-sample quantitative radiomics analysis model capable of successfully distinguishing patients with osteoporosis from patients with osteopenia; the radiomics characteristics can complement existing clinical diagnostic systems and are new strategies and methods for accurately diagnosing osteoporotic and osteopenic conditions in patients.

The invention discloses a model based on machine learning-radiomics and application thereof, which combines the characteristics of the radiomics and clinical risk factors to establish a radiomics Nomogram model to diagnose the conditions of osteoporosis and osteopenia of patients. The diagnosis method can accurately distinct patinets with osteoporosis from patients with osteopenia, thus having great application value for selection of clinical therapeutic regimen.

### BRIEF DESCRIPTION OF THE DRAWINGS

FIG. 1 illustrating the characteristic analysis and index selection by virtue of minimum redundancy and maximum relevance (mRMR) algorithm and least absolute shrinkage and selection operator (LASSO) algorithm; (A) mRMR was performed to eliminate the redundant and irrelevant features; (B) The area under the curve was plotted versus Log (λ). (C) LASSO coefficient profiles of the 20 texture features; A coefficient profile plot was produced against the Log (λ) sequence; Vertical line was drawn at the value selected using 10-fold cross-validation, where optimal λ resulted in 6 nonzero coefficients;

FIG. 2 is the diagram illustrating the 6 radiomics characteristics in rad-score model according to their contribution;

FIG. 3 is the diagram illustrating the analysis results of three diagnosis models' performance in diagnosing osteoporosis and osteopenia, (A) Radiomics model had the potential ability to discriminate the osteoporosis and osteopenia in the training cohort, (B) Radiomics model had the potential ability to discriminate the osteoporosis and osteopenia in the validation cohort;

FIG. 4 is diagram illustrating the visualization of Nomogram model which is the combined model used to diagnose osteoporosis and osteopenia and the performance analysis thereof, wherein (A) Nomogram based on radiomics signatures and clinical factors; (B) Calibration curves of the radiomics nomogram in the training cohort; (C) Calibration curves of the radiomics nomogram in the validation cohorts;

FIG. 5 is the analysis results for the decision curve of the diagnosis models with and without rad-score characteristic;

### DETAILED DESCRIPTION OF THE PREFERRED EMBODIMENTS

The invention is further described in detail hereinafter with reference to the drawings.

(I) Data Collection

Selecting 1120 patients who received QCT tests at the hospital from November 2016 to November 2019. Collecting QCT images of the patients and their clinical examination data such as sex, age, Hemoglobin (HGB), Glucose (GLU), Total Bilirubin (TBIL), Direct Bilirubin (DBIL), Indirect Bilirubin (IBIL), alkaline phosphatase (ALP), Uric Acid (UA), calcium (Ca), magnesium (Mg), phosphorus (P), Homocysteine (HCY) at the same time period as the QCT detection. The exclusion criteria are as follows: 1. fracture of lumbar vertebra or internal fixation of fracture of lumbar vertebra; 2. malignant space-occupying lesion of lumbar vertebra; 3. metabolic or endocrine diseases such as hyperthyroidism or hypothyroidism, space-occupying lesions of thyroid, diabetes, neurological diseases (e.g., parkinson's disease, alzheimer's disease); 4. chronic obstructive pulmonary disease; 5. poor image quality; 6. the clinical characteristics of the missed diagnosis are more than 3 and finally data for 590 patients that meet the requirement has been selected.

grouping patients: 590 patients with osteoporosis and osteopenia were randomly assigned to the training (N=414) and validation cohorts (N=176) (Table 1) with a ratio of 7:3. All the characteristics of the patients in the two cohorts have no statistical difference.

TABLE 1
Characteristics of Patients.

| | Training | | Validation | | |
|---|---|---|---|---|---|
| Characteristics | (N = 414) | | (N = 176) | | P |
| Gender, No. (%) | | | | | |
| Male | 106 | (25.6) | 40 | (22.7) | 0.459 |
| Female | 308 | (74.4) | 136 | (77.3) | |
| Age, mean (SE), years | 66.89 | (0.54) | 66.07 | (0.83) | 0.403 |
| HGB, mean (SE), g/L | 123.77 | (1.30) | 123.77 | (1.79) | 0.999 |
| GLU, mean (SE), mmol/L | 5.46 | (0.07) | 5.57 | (0.12) | 0.418 |
| TBIL, mean (SE), umol/L | 16.06 | (2.81) | 14.61 | (0.56) | 0.737 |
| DBIL, mean (SE), umol/L | 4.24 | (0.67) | 4.15 | (0.27) | 0.929 |
| IBIL, mean (SE), umol/L | 12.58 | (2.89) | 10.47 | (0.38) | 0.635 |
| ALP, mean (SE), U/L | 82.24 | (1.28) | 82.60 | (2.66) | 0.892 |
| UA, mean (SE), umol/L | 302.78 | (4.11) | 304.95 | (6.72) | 0.778 |
| Ca, mean (SE), mmol/L | 2.56 | (0.25) | 2.30 | (0.01) | 0.497 |
| Mg, mean (SE), mmol/L | 1.05 | (0.01) | 1.05 | (0.01) | 0.919 |
| P, mean (SE), mmol/L | 1.10 | (0.009) | 1.09 | (0.01) | 0.509 |
| HCY, mean (SE), umol/L | 13.93 | (0.45) | 13.95 | (0.67) | 0.983 |

NOTE.
P value is derived from the univariable association analyses. Chi-Square was used to analyze the difference of categorical data (Age), while the independent sample t-test was used to analyze the difference of quantitative data (Age, HGB, GLU, TBIL, DBIL, IBIL, ALP, UA, Ca, Mg, P, HCY)
Abbreviations: HGB, hemoglobin; GLU, glucose; TBIL, total bilirubin; DBIL, direct bilirubin; IBIL, indirect bilirubin; ALP, alkaline phosphatase; UA, uric acid; Ca, calcium; Mg, magnesium; P, phosphorus; HCY, homocysteine; SE, standard error.

The patients in the training cohorts and the validation cohorts were divided into an osteopenia group (−2.5<T score <−1), and an osteoporosis group (T score ≤−2.5) according to the current diagnostic T score. The characteristics of patients in each group are shown in Table 2. Age, alkaline phosphatase (ALP), Homocysteine (HCY) are the first three clinical features of the training cohorts with the most significant difference in osteoporosis and osteopenia (P<0.05).

TABLE 2
Characteristics of Patients in the Training and Validation Cohorts.

| | Training Cohorts | | | | | Validation Cohorts | | | | |
|---|---|---|---|---|---|---|---|---|---|---|
| Characteristics | Osteoporosis | | Osteopenia | | P | Osteoporosis | | Osteopenia | | P |
| Gender, No. (%) | | | | | 0.032* | | | | | 0.897 |
| Male | 86 | (28.4) | 20 | (18.0) | | 29 | (22.5) | 11 | (23.4) | |
| Female | 217 | (71.6) | 91 | (82.0) | | 100 | (77.5) | 36 | (76.6) | |
| Age, mean (SE), years | 70.03 | (0.55) | 58.32 | (0.92) | <0.001* | 69.11 | (0.85) | 57.72 | (1.45) | <0.001* |
| HGB, mean (SE), g/L | 122.28 | (1.67) | 127.83 | (1.59) | 0.017* | 125.46 | (1.54) | 127.17 | (2.60) | 0.567 |
| GLU, mean (SE), mmol/L | 5.56 | (0.07) | 5.21 | (0.13) | 0.017* | 5.80 | (0.15) | 5.28 | (0.08) | 0.002* |
| TBIL, mean (SE), umol/L | 17.18 | (3.83) | 13.02 | (0.59) | 0.512 | 14.52 | (0.69) | 14.86 | (0.89) | 0.787 |
| DBIL, mean (SE), umol/L | 4.66 | (0.91) | 3.11 | (0.13) | 0.306 | 4.30 | (0.35) | 3.74 | (0.34) | 0.355 |
| IBIL, mean (SE), umol/L | 13.56 | (3.95) | 9.91 | (0.49) | 0.576 | 10.22 | (0.45) | 11.17 | (0.70) | 0.262 |
| ALP, mean (SE), U/L | 84.49 | (1.50) | 76.13 | (2.40) | 0.004* | 85.06 | (3.36) | 75.84 | (3.64) | 0.126 |
| UA, mean (SE), umol/L | 301.77 | (4.69) | 305.54 | (8.48) | 0.685 | 307.97 | (8.39) | 296.66 | (10.19) | 0.458 |
| Ca, mean (SE), mmol/L | 2.64 | (0.34) | 2.34 | (0.01) | 0.598 | 2.29 | (0.01) | 2.33 | (0.02) | 0.049* |
| Mg, mean (SE), mmol/L | 1.06 | (0.01) | 1.04 | (0.02) | 0.370 | 1.07 | (0.02) | 1.01 | (0.02) | 0.058 |
| P, mean (SE), mmol/L | 1.09 | (0.01) | 1.13 | (0.02) | 0.047* | 1.09 | (0.01) | 1.10 | (0.02) | 0.858 |
| HCY, mean (SE). umol/L | 14.83 | (0.58) | 11.48 | (0.50) | <0.001* | 14.90 | (0.86) | 12.83 | (0.71) | 0.168 |

NOTE.
P value is derived from the univariable association analyses. Chi-Square was used to analyze the difference of categorical data (Age), while the independent sample t-test was used to analyze the difference of quantitative data (Age, HGB, GLU, TBIL, DBIL, IBIL, ALP, UA, Ca, Mg, P, HCY)
Abbreviations: HGB, hemoglobin; GLU, glucose; TBIL, total bilirubin; DBIL, direct bilirubin; IBIL, indirect bilirubin; ALP, alkaline phosphatase; UA, uric acid; Ca, calcium; Mg, magnesium; P, phosphorus; HCY, homocysteine; SE, standard error.
*P value < 0.05.

(II) Radiomics Data Processing

Region of interest (ROI) segmentation and feature extraction: under the guidance of a professional radiologist, a third lumbar vertebra (L3) cancellous bone image selected uniformly, two medical students use 3D slicer software (version 4.10.2; www.slicer.org) to manually segment the ROI on the CT image, and extract 851 radiomics quantitative characteristics. These characteristics include shape, gray level dependence matrix (gldm), gray level co-occurrence matrix (glcm), initial order, gray level run length matrix (glrlm), gray level size zone matrix (glszm), neighboring gray-tone difference matrix (ngtdm).

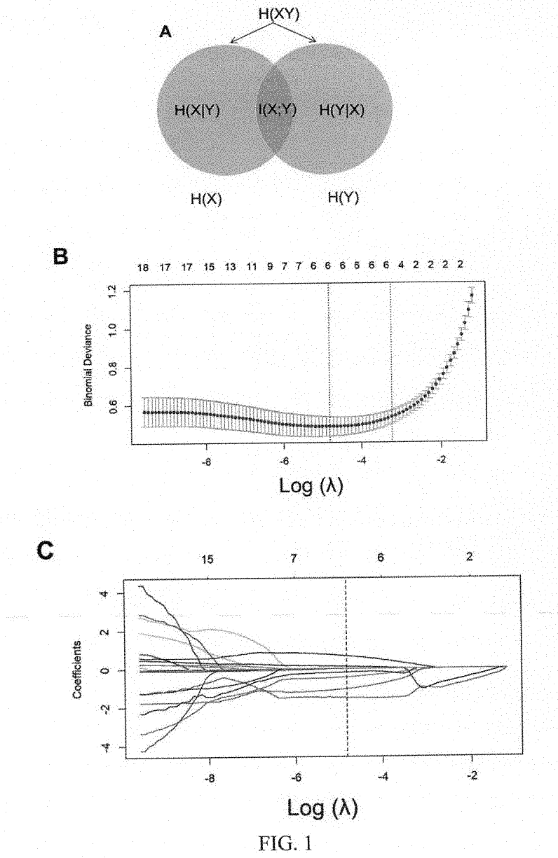

(III) Realizing machine learning minimum redundancy and maximum relevance (mRMR) algorithm and least absolute shrinkage and selection operator (LASSO) algorithm via “mRMRe” and “glmnet” packet in R software; firstly, 20 potential predictors are selected from 851 radiomics characteristics via the minimum redundancy and maximum relevance (mRMR) algorithm (FIG. 1A); then the optimal characteristics of 6 non-zero. coefficients are further selected by using least absolute shrinkage and selection operator (LASSO) algorithm (FIGS. 1B and C) which comprise wavelet.LLL.firstorder.10Percentile, wavelet.LLH.firstorder.InterquartileRange, original.firstorder.10Percentile, wavelet.LLL.first-order.Median, wavelet.LLH.glrlm.ShortRunGrayLevelEmphasis, and wavelet.LLH.firstorder.-Skewness.

At the same time, selecting candidate factors of clinical information by logistic regression; and screening out 3 clinical characteristics: Age, alkaline phosphatase (ALP), and homocysteine (HCY);

2. Establishment of the Radiomics Score (Rad-Score) Model

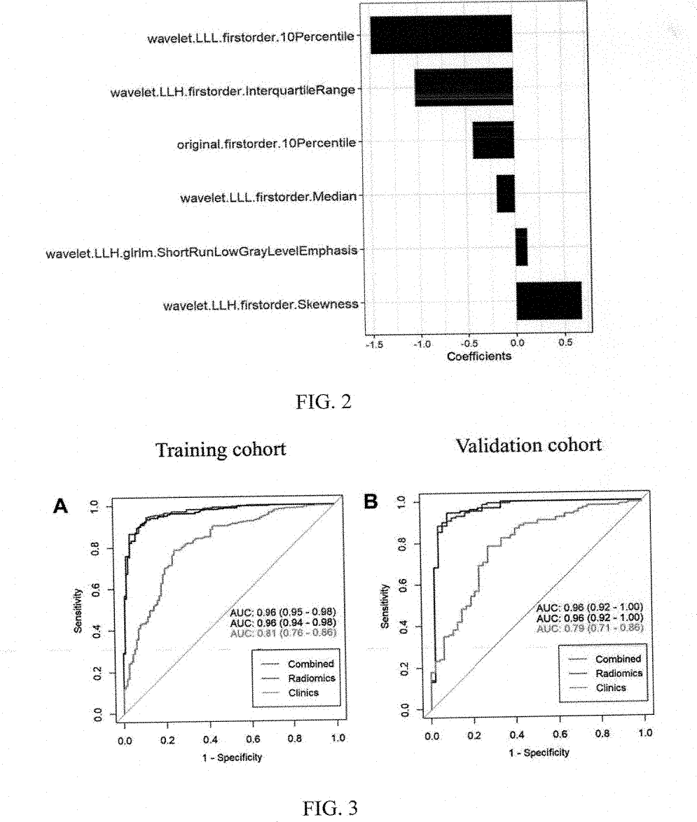

Combining the selected 6 radiomics characteristics according to contribution weighting, and developing rad-score model (FIG. 2), Rad-score=−1.486*wavelet.LLL.firstorder.10Percentile+−1.032*wavelet.LLH.firstorder.Interquartile Range+−0.428*original.firstorder.10Percentile+−0.187*wavelet.LLL.firstorder.Median+0.123*wavelet.LLH.glrlm.ShortRunLowGrayLevelEmphasis+0.68*wavelet.LLH.firstorder. Skewness+2.755;

3. Establishment and Comparison of Diagnosis Models

Establishing 3 diagnosis models according to the radiomics characteristics and clinical variables, and comparing the performances of the models in the diagnosis of osteoporosis and osteopenia (FIG. 3).

Clinics model, built merely by 3 clinical characteristics i.e. Age, alkaline phosphatase (ALP), and homocysteine (HCY), and the AUC in the training cohorts is 0.81 (95% CI, 0.78-0.86) while the AUC in the validation cohorts is 0.79 (95% CI, 0.71-0.86);

Radiomics model, built merely by rad-score and the AUC in the training cohorts is 0.96 (95% CI, 0.94-0.98) while the AUC in the validation cohorts is 0.96 (95% CI, 0.92-1.00);

Combined model, built by combination of rad-score and 3 clinical characteristics (Age, ALP, HCY) wherein the AUC in the training cohorts is 0.96 (95% CI, 0.95-0.98) while the AUC in the validation cohorts is 0.96 (95% CI, 0.92-1.00);

The accuracy, sensitivity and specificity of the three models in diagnosing osteoporosis and osteopenia are compared at the same time. The results show that the performance of the radiomics model and the combined model is very good whether in the training cohorts or the validation cohorts, and the performance of the combined model has the best performance compared with the former two models in terms of diagnostic sensitivity, thereby being beneficial to early discovery of osteoporosis (Table 3).

TABLE 3
Diagnostic performance of with or without radiomics.

| | | | Sensi- | Speci- | | |
|---|---|---|---|---|---|---|
| Group | Model | Accuracy | tivity | ficity | PPV | NPV |
| Training | Clinics | 0.778 | 0.898 | 0.564 | 0.785 | 0.757 |
| Validation | Clinics | 0.739 | 0.881 | 0.507 | 0.744 | 0.723 |
| Training | Radiomics | 0.903 | 0.928 | 0.894 | 0.763 | 0.971 |
| Validation | Radiomics | 0.938 | 0.915 | 0.946 | 0.860 | 0.968 |
| Training | Nomogram | 0.891 | 0.985 | 0.723 | 0.865 | 0.964 |
| Validation | Nomogram | 0.903 | 0.983 | 0.750 | 0.884 | 0.957 |

Abbreviations: PPV, positive predictive value;
NPV, negative predictive value

4. Application of the Combined Model

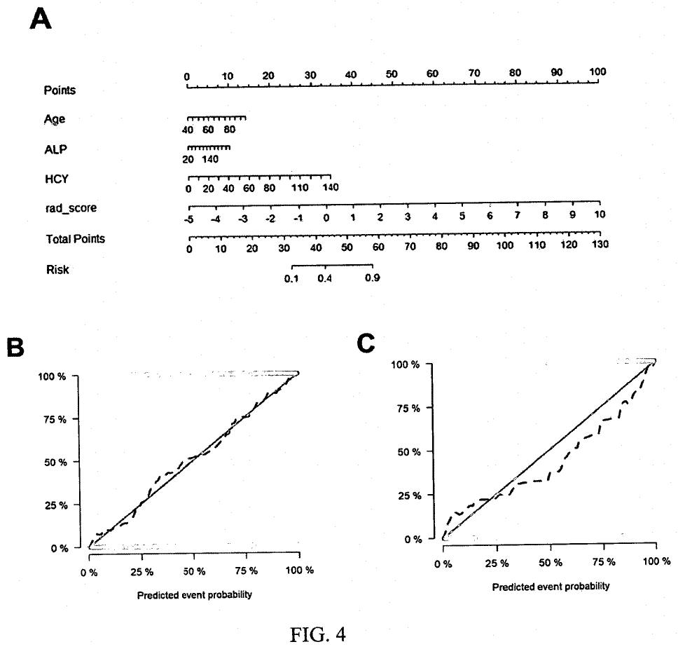

By comparison, the combined model comprising rad-score and 3 clinical characteristics (Age, ALP, HCY) has the best performance in diagnosing osteoporosis and osteopenia. To facilitate the clinical application and simplify the operation, performing visualization processing on the combined model by using the “rms” packet in the R software to obtain the Nomogram model, as shown in FIG. 4A; the calibration curves for this model show good consistency between the predictive values and the actual observed values in the training and validation cohorts (FIGS. 4B and C).

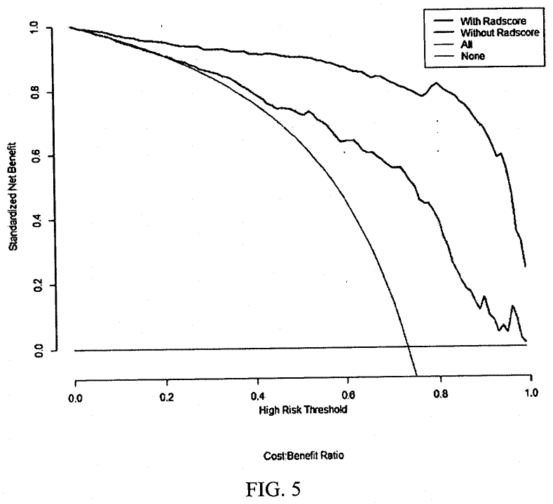

Comparing the decision curves of the diagnostic models with and without the rad-score characteristics and the results are shown in FIG. 5; the net benefit of Nomogram model with the rad-score model is higher in identifying osteoporosis and osteopenia than the model without the rad-score model. It can be seen that the diagnosis model constructed based on the radiomics characteristics has good application value in identifying osteoporosis and osteopenia.

The invention and its embodiments have been described above, but the description is not limited thereto; only one embodiment of the invention is shown in the drawings, and the actual structure is not limited thereto. In general, it is to be understood by those skilled in the art that non-creative design of structural forms and embodiments that are similar to the technical solutions without departing from the spirit of the invention shall all fall within the protective scope of the invention.

1. A model based on machine learning-radiomics wherein the model is the Nomogram model;
the establishment of said Nomogram model comprises following steps:
S1. collecting data and predicting factor selection: selecting case samples, and processing data by using machine learning minimum redundancy and maximum relevance (mRMR) algorithm and least absolute shrinkage and selection operator (LASSO) algorithm via “mRMRe” and “glmnet” packet in R software;
20 potential predictors are selected from 851 radiomics characteristics via the minimum redundancy and maximum relevance (mRMR) algorithm;
the optimal characteristics of 6 non-zero coefficients are further selected by using least absolute shrinkage and selection operator (LASSO) algorithm;
selecting candidate factors of clinical information by logistic regression, and screening out 3 clinical characteristics: Age, alkaline phosphatase (ALP), and homocysteine (HCY);
S2. combining the selected 6 radiomics characteristics according to contribution weighting, and developing the rad-score model;
S3. establishing different diagnosis models, and comparing the performances of the diagnosis models in the diagnosis of osteoporosis and osteopenia, wherein said diagnosis models comprise:
Clinics model, built merely by 3 clinical characteristics (Age, ALP, HCY), and the AUC in the training cohorts is 0.81 (95% CI, 0.78-0.86) while the AUC in the validation cohorts is 0.79 (95% CI, 0.71-0.86);
Radiomics model, built merely by rad-score and the AUC in the training cohorts is 0.96 (95% CI, 0.94-0.98) while the AUC in the validation cohorts is 0.96 (95% CI, 0.92-1.00);
Combined model, built by combination of rad-score and 3 clinical characteristics (Age, ALP, HCY) wherein the AUC in the training cohorts is 0.96 (95% CI, 0.95-0.98) while the AUC in the validation cohorts is 0.96 (95% CI, 0.92-1.00);
S4. performing visualization processing on the combined model by using the “rms” packet in the R software to obtain the Nomogram model;
S5. Validation of the Nomogram model.

2. The model based on machine learning-radiomics of claim 1, wherein case samples in step S1 are more than or equal to 300 cases; the cases' selection adopts an exclusive method, and the exclusion criteria are as follows: a. fracture of lumbar vertebra or internal fixation of fracture of lumbar vertebra; b. malignant space-occupying lesion of lumbar vertebra; c. metabolic or endocrine diseases such as hyperthyroidism or hypothyroidism, space-occupying lesions of thyroid, diabetes, neurological diseases (e.g., parkinson's disease, alzheimer's disease); d. chronic obstructive pulmonary disease; e. poor image quality; f. the clinical characteristics of the missed diagnosis are more than 3.

3. An application of said model based on machine learning-radiomics of claim 1-2, wherein it is used for diagnosing and identifying osteoporosis and osteopenia.
